(19) United States
(12) Patent Application Publication
(10) Pub. No.: US 2026/0212505 A1
(43) Pub. Date: Jul. 23, 2026

(54) AUTOMATED ANALYSIS OF ECHOCARDIOGRAPHIC IMAGES

(71) Applicants: AISAP LTD, Ramat Gan (IL); Sheba Impact Ltd., Ramat Gan (IL)

(72) Inventors: Adiel Amshalom, Tel Aviv (IL); Michael Fiman, Rosh Haayin (IL); Robert Klempfner, Hertzeliya (IL); Ehud Schwammenthal, Raanana (IL); Elad Maor, Tel Aviv (IL)

(21) Appl. No.: 19/137,498

(22) PCT Filed: Dec. 11, 2023
(86) PCT No.: PCT/IB2023/062471

§ 371 Date: Jun. 10, 2025

Related U.S. Application Data
(60) Provisional application No. 63/432,230, filed on Dec. 13, 2022.

(51) Int. Cl. G06T 7/00; A61B 8/08; G06V 10/62; G06V 10/764; G16H 50/20

(52) U.S. Cl. CPC G06T 7/0016; A61B 8/0883; G06V 10/62; G06V 10/764; G16H 50/20; G06T 2207/10132; G06T 2207/20212; G06T 2207/30048; G06V 2201/031

(57) ABSTRACT

Apparatus and methods are described, including receiving a stream of echocardiographic images of a patient's heart that are acquired from a plurality of different views, analyzing the stream of echocardiographic images, and determining a cardiac-state age of the patient based upon the analysis, the cardiac-state age of the patient being indicative of an age that the patient's heart appears to be, based upon the analysis of the stream of echocardiographic images. Other applications are also described.

### CROSS-REFERENCES TO RELATED APPLICATIONS

The present application claims priority from U.S. Provisional Patent Application 63/432,230 to Amshalom et al., entitled “Automated analysis of echocardiographic images,” filed Dec. 13, 2022, which is incorporated herein by reference.

### FIELD OF EMBODIMENTS OF THE INVENTION

Some applications of the present invention generally relate to medical apparatus and methods. Specifically, some applications of the present invention relate to apparatus and methods for performing automated analysis of echocardiographic images.

### BACKGROUND

Ultrasound is a widely used imaging modality, having advantages over certain other imaging modalities in that it is non-radiative, it is non-invasive, and the required equipment is relatively cheap. Echocardiography utilizes ultrasound for imaging the heart and is the most commonly used examination for the diagnosis of clinical conditions in cardiology, with 35 million such examination being performed annually in the USA. Echocardiography is used to measure the size and shape of the heart, to identify and assess tissue damage, valves, and other structures of the heart. Echocardiograms are also used to estimate parameters relating to cardiac function, such as cardiac output and ejection fraction.

Typically, echocardiograms taken from respective anatomical views of interest are identified by a healthcare professional (e.g., a technician and/or a physician) and the parameters are then extracted from the echocardiograms by the healthcare professional making measurements on the identified echocardiograms. The manual nature of this analysis is time consuming and requires highly qualified personnel, which often leads to delayed and missed diagnoses and high variability in the interpretation of echocardiograms.

### SUMMARY

In accordance with some applications of the present invention, an automated echocardiographic image analysis system includes a plurality of ultrasound transducers and local computer processors, as well as one or more remote computer processors, the computer processors being in communication with each other via a network. Typically, a user acquires echocardiographic images of a patient using the ultrasound transducer. For some applications, the user manually inputs data regarding the patient and/or extracts data regarding the patient (e.g., from the patient's medical records) for use by the system, using the user interface device. The patient's echocardiographic images are analyzed by one or more of the computer processors, using the analysis techniques described herein. For some applications, based upon the analysis of the patient's echocardiographic images, an output is generated via the user interface device. For example, parameters of the patient that are determined based on the echocardiographic images are outputted, a diagnosis of the patient is outputted, and/or predictions regarding the patient based on the echocardiographic images may be outputted.

Using prior art techniques it is typically the case that a technician is required to acquire echocardiographic image loops from respective views and then select the optimal echocardiographic image frames from the echocardiographic image loops for performing measurements on the patient's heart. Typically, using the techniques described herein, an entire stream of echocardiographic images, which are acquired from a plurality of different views, is provided as an input to the system. The system is trained to automatically identify image loops that correspond to respective views, to select optimum images and/or image loops for analysis, to extract data from the images and/or image loops, to identify patterns in the data, to extract parameters relating to the patient, and/or to generate predictions regarding patients. Typically, during an initial training stage, the computer processors are trained to perform the above-described steps using video streams of echocardiographic images of many patients. In a subsequent analysis stage, the computer processors are configured to apply patterns that were learned during the training stage to the analysis of a video stream of echocardiographic images of a particular patient in order to perform the above-described steps with respect to the patient. Typically, the echocardiographic images of at least some of (and typically all of) the patients whose echocardiographic images are analyzed by the system in the analysis stage are also utilized for ongoing training of the system. In this manner, training of the system is continuous and is improved over time as the system continues to be provided with additional echocardiographic images and data.

For some applications, the system determines that the patient is suffering from one or more of myocardial infarction, an aortic aneurysm, amyloidosis, dilated cardiomyopathy, hypertrophic cardiomyopathy, pulmonary hypertension, pulmonary emboli, atherosclerosis, aortic stenosis, mitral regurgitation, aortic regurgitation, left ventricular enlargement, and/or left ventricular hypertrophy. For some applications, the system determines that the patient is a candidate for a valve intervention, or an alternative treatment. For some applications, the system determines one or more cardiac or cardiac-related parameters, such as left ventricular ejection fraction, left ventricular function, interventricular septum thickness, left ventricular diastolic function, right ventricular function, and/or right ventricular diastolic function. Alternatively or additionally, the system predicts the likelihood of the patient undergoing a clinical episode. Further alternatively or additionally, the system determines that the patient has a condition that affects a portion of the patient's body other than the patient's heart (such as a renal condition, a hepatic condition, a lymphatic condition, ovarian cancer etc.) , by analyzing the patient's echocardiographic images.

For some applications, based upon the analysis of echocardiographic images, the system determines a cardiac-state age of the patient, the cardiac-state age of the patient being indicative of a risk of the patient suffering from cardiac conditions. For some applications, the system receives an input indicating the patient's chronological age, and makes a prediction regarding the patient based upon a difference between the patient's chronological age and the patient's cardiac-state age. For some applications, based upon the analysis of echocardiographic images, the system determines a cardiac-state sex of the patient, the cardiac-state sex of the patient being indicative of a risk of the patient suffering from cardiac conditions. For some applications, the system receives an input indicating the patient's biological sex, and makes a prediction regarding the patient based upon a difference between the patient's biological sex and the patient's cardiac-state sex.

Although some applications of the present disclosure have been described as being applied to echocardiographic images, the scope of the disclosure includes applying generally similar techniques to echo images of a different portion of the subject's anatomy (e.g., the subject's lungs, reproductive system, gastrointestinal tract, etc.) and/or to images acquired in a different imaging modality (such as MRI, X-ray, CT, PET, etc.), mutatis mutandis.

There is therefore provided, in accordance with some embodiments of the present invention, apparatus including:
at least one computer processor configured to:
   receive a stream of echocardiographic images of a patient's heart that are acquired from a plurality of different views;
   analyze the stream of echocardiographic images; and
   determine a cardiac-state age of the patient based upon the analysis, the cardiac-state age of the patient being indicative of an age that the patient's heart appears to be, based upon the analysis of the stream of echocardiographic images.

In some embodiments, the at least one computer processor is configured to receive an input indicating the patient's chronological age, and the computer processor is configured to make a prediction regarding the patient based upon a difference between the patient's chronological age and the patient's cardiac-state age.

In some embodiments, in response to determining that it is not possible to determine the cardiac-state age of the patient based upon the analysis a given degree of certainty, the computer processor is configured to generate an output indicating that this is the case.

In some embodiments, the at least one computer processor is configured to diagnose the patient and/or make a prediction regarding the patient at least partially based upon the analysis of the stream of echocardiographic images.

In some embodiments, the at least one computer processor is configured to:
analyze the stream of echocardiographic images using a visual-transformer algorithm;
diagnose the patient and/or make a prediction regarding the patient based upon the analysis; and
generate an output indicating the diagnosis and/or prediction, and indicating which features and/or patterns within the stream of echocardiographic images of the patient's heart that led to the diagnosis and/or prediction.

In some embodiments, the at least one computer processor is configured to:
classify the echocardiographic images and/or loops of the echocardiographic images as corresponding to respective views;
perform further analysis of a portion of the echocardiographic images and/or echocardiographic image loops that correspond to one more views; and
determine the cardiac-state age of the patient based upon the based upon the further analysis of the portion of the echocardiographic images and/or echocardiographic image loops that correspond to one more views.

In some embodiments, at least some of the echocardiographic images and/or echocardiographic image loops that correspond to one more views that are selected for the further analysis are randomly selected.

In some embodiments, at least some of the echocardiographic images and/or echocardiographic image loops are randomly removed from being used for the further analysis of the portion of the echocardiographic images and/or echocardiographic image loops that correspond to one more views.

In some embodiments, the at least one computer processor is configured to:
select one or more echocardiographic images and/or echocardiographic image loops within the stream of echocardiographic images;
for each of the one or more selected echocardiographic images and/or echocardiographic image loops, perform multi-target analysis by extracting a plurality of features from the one or more echocardiographic images and/or echocardiographic image loops in parallel with each other; and
diagnose the patient and/or make a prediction regarding the patient at least partially based upon the plurality of features that are extracted from the one or more echocardiographic images and/or echocardiographic image loops.

In some embodiments, the at least one computer processor is configured to:
derive features from individual echocardiographic images;
combine features from temporally proximate echocardiographic images within a loop of echocardiographic images such that changes over time are extracted from the loop of echocardiographic images; and
diagnose the patient and/or make a prediction regarding the patient at least partially based upon the changes over time that are extracted from the loop of echocardiographic images.

In some embodiments, the at least one computer processor is configured to define a plurality of loops of echocardiographic images with each of the loops starting from a different starting point, and the at least one computer processor is configured to combine features from temporally proximate echocardiographic images within each of the plurality of loops of echocardiographic images.

In some embodiments, the at least one computer processor is configured to determine a cardiac-state sex of the patient, the cardiac-state sex of the patient being indicative of a sex that the patient appears to be based upon the analysis of the of stream of echocardiographic images.

In some embodiments, the computer processor is configured to receive an input indicating the patient's biological sex, and the computer processor is configured to make a prediction regarding the patient based upon a difference between the patient's biological sex and the patient's cardiac-state sex.

There is further provided, in accordance with some embodiments of the present invention, a method including:
using at least one computer processor:
   receiving a stream of echocardiographic images of a patient's heart that are acquired from a plurality of different views;
   analyzing the stream of echocardiographic images; and
   determining a cardiac-state age of the patient based upon the analysis, the cardiac-state age of the patient being indicative of an age that the patient's heart appears to be, based upon the analysis of the stream of echocardiographic images.

There is further provided, in accordance with some embodiments of the present invention, a computer software product, for use with a blood sample, the computer software product including a non-transitory computer-readable medium in which program instructions are stored, which instructions, when read by a computer cause the computer to perform the steps of:
receiving a stream of echocardiographic images of a patient's heart that are acquired from a plurality of different views;
analyzing the stream of echocardiographic images; and
determining a cardiac-state age of the patient based upon the analysis, the cardiac-state age of the patient being indicative of an age that the patient's heart appears to be, based upon the analysis of the stream of echocardiographic images.

There is further provided, in accordance with some embodiments of the present invention, apparatus including:
at least one computer processor configured to:
   receive a stream of echocardiographic images of a patient's heart that are acquired from a plurality of different views;
   classify echocardiographic images and/or loops of the echocardiographic images as corresponding to respective views;
   perform further analysis of a portion of the echocardiographic images and/or echocardiographic image loops that correspond to one more views, at least some of the echocardiographic images and/or echocardiographic image loops that correspond to one more views that are selected for the further analysis being randomly selected; and
   diagnose the patient and/or make a prediction regarding the patient at least partially based upon the further analysis of the portion of the echocardiographic images and/or echocardiographic image loops that correspond to one more views.

In some embodiments, in response to determining that it is not possible to diagnose the patient and/or make a prediction regarding the patient to a given degree of certainty, the computer processor is configured to generate an output indicating that this is the case.

In some embodiments, at least some of the echocardiographic images and/or echocardiographic image loops are randomly removed from being used for the further analysis of the portion of the echocardiographic images and/or echocardiographic image loops that correspond to one more views.

In some embodiments, the at least one computer processor is configured to:
select one or more echocardiographic images and/or echocardiographic image loops from within the stream of echocardiographic images;
for each of the one or more selected echocardiographic images and/or echocardiographic image loops, perform multi-target analysis by extracting a plurality of features from the one or more echocardiographic images and/or echocardiographic image loops in parallel with each other; and
diagnose the patient and/or make a prediction regarding the patient at least partially based upon the plurality of features that are extracted from the one or more echocardiographic images and/or echocardiographic image loops.

In some embodiments, the at least one computer processor is configured to:
analyze the stream of echocardiographic images using a visual-transformer algorithm;
diagnose the patient and/or make a prediction regarding the patient based upon the analysis; and
generate an output indicating the diagnosis and/or prediction, and indicating which features and/or patterns within the stream of echocardiographic images of the patient's heart that led to the diagnosis and/or prediction.

In some embodiments, the at least one computer processor is configured to:
derive features from individual echocardiographic images;
combine features from temporally proximate echocardiographic images within a loop of echocardiographic images such that changes over time are extracted from the loop of echocardiographic images; and
diagnose the patient and/or make a prediction regarding the patient at least partially based upon the changes over time that are extracted from the loop of echocardiographic images.

In some embodiments, the at least one computer processor is configured to define a plurality of loops of echocardiographic images, with each of the loops starting from a different starting point, and the at least one computer processor is configured to combine features from temporally proximate echocardiographic images within each of the plurality of loops of echocardiographic images.

In some embodiments, the at least one computer processor is configured to determine a cardiac-state age of the patient, the cardiac-state age of the patient being indicative of an age that the patient's heart appears to be, based upon the further analysis of the portion of the echocardiographic images and/or echocardiographic image loops that correspond to one more views.

In some embodiments, the computer processor is configured to receive an input indicating the patient's chronological age, and the computer processor is configured to make a prediction regarding the patient based upon a difference between the patient's chronological age and the patient's cardiac-state age.

In some embodiments, the at least one computer processor is configured to determine a cardiac-state sex of the patient, the cardiac-state sex of the patient being indicative of a sex that the patient appears to be, based upon the further analysis of the portion of the echocardiographic images and/or echocardiographic image loops that correspond to one more views.

In some embodiments, the computer processor is configured to receive an input indicating the patient's biological sex, and the computer processor is configured to make a prediction regarding the patient based upon a difference between the patient's biological sex and the patient's cardiac-state sex.

There is further provided, in accordance with some embodiments of the present invention, a method including:
using at least one computer processor:
   receiving a stream of echocardiographic images of a patient's heart that are acquired from a plurality of different views;
   classifying echocardiographic images and/or loops of the echocardiographic images as corresponding to respective views;
   performing further analysis of a portion of the echocardiographic images and/or echocardiographic image loops that correspond to one more views, at least some of the echocardiographic images and/or echocardiographic image loops that correspond to one more views that are selected for the further analysis being randomly selected; and
   diagnosing the patient and/or making a prediction regarding the patient at least partially based upon the further analysis of the portion of the echocardiographic images and/or echocardiographic image loops that correspond to one more views.

There is further provided, in accordance with some embodiments of the present invention, a computer software product, for use with a blood sample, the computer software product including a non-transitory computer-readable medium in which program instructions are stored, which instructions, when read by a computer cause the computer to perform the steps of:
receiving a stream of echocardiographic images of a patient's heart that are acquired from a plurality of different views;
classifying echocardiographic images and/or loops of the echocardiographic images as corresponding to respective views;
performing further analysis of a portion of the echocardiographic images and/or echocardiographic image loops that correspond to one more views, at least some of the echocardiographic images and/or echocardiographic image loops that correspond to one more views that are selected for the further analysis being randomly selected; and
diagnosing the patient and/or making a prediction regarding the patient at least partially based upon the further analysis of the portion of the echocardiographic images and/or echocardiographic image loops that correspond to one more views.

There is further provided, in accordance with some embodiments of the present invention, apparatus including:
at least one computer processor configured to:
   receive a stream of echocardiographic images of a patient's heart that are acquired from a plurality of different views;
   derive features from individual echocardiographic images;
   combine features from temporally proximate echocardiographic images within a loop of echocardiographic images such that changes over time are extracted from the loop of echocardiographic images; and
   diagnose the patient and/or make a prediction regarding the patient at least partially based upon the changes over time that are extracted from the loop of echocardiographic images.

In some embodiments, the at least one computer processor is configured to define a plurality of loops of echocardiographic images, with each of the loops starting from a different starting point, and wherein the at least one computer processor is configured to combine features from temporally proximate echocardiographic images within each of the plurality of loops of echocardiographic images.

In some embodiments, in response to determining that it is not possible to diagnose the patient and/or make a prediction regarding the patient to a given degree of certainty, the computer processor is configured to generate an output indicating that this is the case.

In some embodiments, the at least one computer processor is configured to:
analyze the stream of echocardiographic images using a visual-transformer algorithm;
diagnose the patient and/or make a prediction regarding the patient based upon the analysis; and
generate an output indicating the diagnosis and/or prediction, and indicating which features and/or patterns within the stream of echocardiographic images of the patient's heart that led to the diagnosis and/or prediction.

In some embodiments, the at least one computer processor is configured to:
classify the echocardiographic images and/or loops of the echocardiographic images as corresponding to respective views;
analyze a portion of the echocardiographic images and/or echocardiographic image loops that correspond to one more views; and
diagnose the patient and/or make a prediction regarding the patient at least partially based upon analyzing the portion of the echocardiographic images and/or echocardiographic image loops that correspond to one more views.

In some embodiments, at least some of the echocardiographic images and/or echocardiographic image loops that correspond to one more views that are selected for analyzing are randomly selected.

In some embodiments, at least some of the echocardiographic images and/or echocardiographic image loops are randomly removed from being used for analyzing the portion of the echocardiographic images and/or echocardiographic image loops that correspond to one more views.

In some embodiments, the at least one computer processor is configured to:
select one or more echocardiographic images and/or echocardiographic image loops within the stream of echocardiographic images;
for each of the one or more selected echocardiographic images and/or echocardiographic image loops, perform multi-target analysis by extracting a plurality of features from the one or more echocardiographic images and/or echocardiographic image loops in parallel with each other; and
diagnose the patient and/or make a prediction regarding the patient at least partially based upon the plurality of features that are extracted from the one or more echocardiographic images and/or echocardiographic image loops.

In some embodiments, the at least one computer processor is configured to determine a cardiac-state age of the patient, the cardiac-state age of the patient being indicative of an age that the patient's heart appears to be based upon the changes over time that are extracted from the loop of echocardiographic images.

In some embodiments, the computer processor is configured to receive an input indicating the patient's chronological age, and the computer processor is configured to make a prediction regarding the patient based upon a difference between the patient's chronological age and the patient's cardiac-state age.

In some embodiments, the at least one computer processor is configured to determine a cardiac-state sex of the patient, the cardiac-state sex of the patient being indicative of a sex that the patient appears to be based upon the changes over time that are extracted from the loop of echocardiographic images.

In some embodiments, the computer processor is configured to receive an input indicating the patient's biological sex, and the computer processor is configured to make a prediction regarding the patient based upon a difference between the patient's biological sex and the patient's cardiac-state sex.

There is further provided, in accordance with some embodiments of the present invention, a method including:
using at least one computer processor:
   receiving a stream of echocardiographic images of a patient's heart that are acquired from a plurality of different views;
   deriving features from individual echocardiographic images;
   combining features from temporally proximate echocardiographic images within a loop of echocardiographic images such that changes over time are extracted from the loop of echocardiographic images; and
   diagnosing the patient and/or making a prediction regarding the patient at least partially based upon the changes over time that are extracted from the loop of echocardiographic images.

There is further provided, in accordance with some embodiments of the present invention, a computer software product, for use with a blood sample, the computer software product including a non-transitory computer-readable medium in which program instructions are stored, which instructions, when read by a computer cause the computer to perform the steps of:
receiving a stream of echocardiographic images of a patient's heart that are acquired from a plurality of different views;
deriving features from individual echocardiographic images;
combining features from temporally proximate echocardiographic images within a loop of echocardiographic images such that changes over time are extracted from the loop of echocardiographic images; and
diagnosing the patient and/or making a prediction regarding the patient at least partially based upon the changes over time that are extracted from the loop of echocardiographic images.

There is further provided, in accordance with some embodiments of the present invention, apparatus including:
at least one computer processor configured to:
   receive a stream of echocardiographic images of the patient's heart that are acquired from a plurality of different views;
   classify echocardiographic images and/or loops of the echocardiographic images as corresponding to respective views;
   perform further analysis of a portion of the echocardiographic images and/or echocardiographic image loops that correspond to one more views, at least some of the echocardiographic images and/or echocardiographic image loops being randomly removed from being used for the further analysis of the portion of the echocardiographic images and/or echocardiographic image loops that correspond to one more views; and
   diagnose the patient and/or make a prediction regarding the patient at least partially based upon the further analysis of the portion of the echocardiographic images and/or echocardiographic image loops that correspond to one more views.

There is further provided, in accordance with some embodiments of the present invention, apparatus including:
at least one computer processor configured to:
   receive a stream of echocardiographic images of the patient's heart that are acquired from a plurality of different views;
   select one or more echocardiographic images and/or echocardiographic image loops within the stream of echocardiographic images; and
   for each of the one or more selected echocardiographic images and/or echocardiographic image loops, perform multi-target analysis by extracting a plurality of features from the one or more echocardiographic images and/or echocardiographic image loops in parallel with each other; and
   diagnose the patient and/or make a prediction regarding the patient at least partially based upon the plurality of features that are extracted from the one or more echocardiographic images and/or echocardiographic image loops.

There is further provided, in accordance with some embodiments of the present invention, apparatus including:
at least one computer processor configured to:
   receive a stream of echocardiographic images of the patient's heart that are acquired from a plurality of different views;
   analyze the stream of echocardiographic images using a visual-transformer algorithm;
   diagnose the patient and/or make a prediction regarding the patient based upon the analysis; and
   generate an output indicating the diagnosis and/or prediction, and indicating which features and/or patterns within the stream of echocardiographic images of the patient's heart that led to the diagnosis and/or prediction.

There is further provided, in accordance with some embodiments of the present invention, apparatus including:
at least one computer processor configured to:
   receive a stream of echocardiographic images of the patient's heart that are acquired from a plurality of different views;
   analyze the stream of echocardiographic images; and
   determine a cardiac-state sex of the patient based upon the analysis, the cardiac-state sex of the patient being indicative of a sex that the patient appears to be, based upon the analysis of the of stream of echocardiographic images.

In some embodiments, the computer processor is configured to receive an input indicating the patient's biological sex, and the computer processor is configured to make a prediction regarding the patient based upon a difference between the patient's biological sex and the patient's cardiac-state sex.

The present invention will be more fully understood from the following detailed description of embodiments thereof, taken together with the drawings, in which:

### BRIEF DESCRIPTION OF THE DRAWINGS

FIGS. 1A, 1B and 1C are examples of echocardiographic images that are analyzed in accordance with some applications of the present invention;

FIG. 2 is a block diagram showing portions of an automated echocardiographic image analysis system, in accordance with some applications of the present invention;

FIG. 3 is a flowchart showing steps that are performed during a training stage, in accordance with some applications of the present invention;

FIG. 4 is a flowchart showing echocardiographic image analysis steps that are performed in an analysis stage, in accordance with some applications of the present invention; and

FIG. 5 is a graph with curves indicating (a) survival rates of patients whose cardiac-state age differed from their chronological age by more than 5 years, and (b) survival rates of patients whose cardiac-state age did not differ from their chronological age by more than 5 years, as determined in accordance with some applications of the present invention.

### DETAILED DESCRIPTION OF EMBODIMENTS

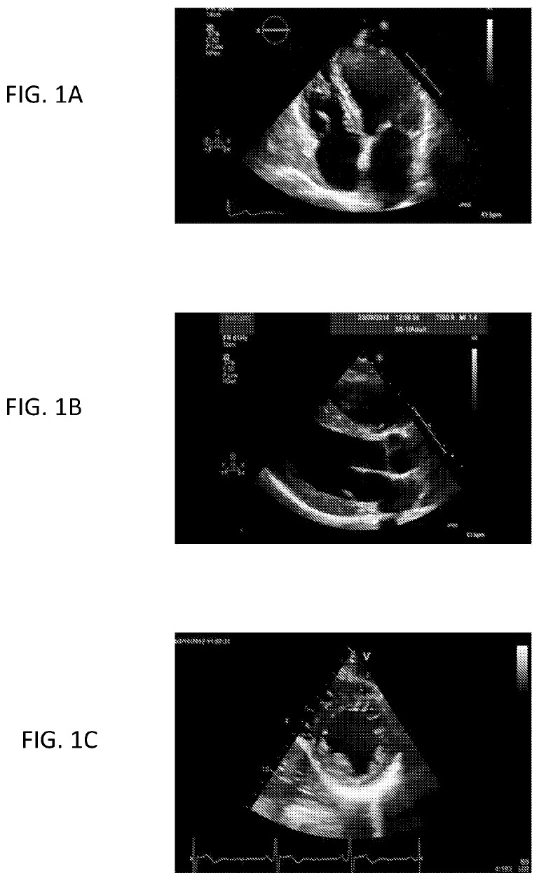

Reference is now made to FIGS. 1A, 1B and 1C are examples of echocardiographic images that are analyzed in accordance with some applications of the present invention. FIG. 1A shows an example of an echocardiographic image that is acquired from the apical window (which is typically acquired from a lateral location in the 4th-5th intercostal space), and specifically is an example of an apical four-chamber view, in which all four chambers of the patient's heart are visible. FIG. 1B shows a parasternal long-axis view (which is typically acquired with the ultrasound transducer positioned in the 3rd or 4th intercostal space on the left side right next to the sternum), in which the interventricular septum, the aortic root and the left atrium are typically visible. FIG. 1C shows a parasternal short-axis view (which is typically acquired by rotating the ultrasound transducer 90 degrees in a clockwise direction from the parasternal long-axis position), in which the aorta is typically visible in the center of the echocardiographic image, and is surrounded by other portions of the cardiac anatomy. For some applications, one or more of the views shown in FIGS. 1A-C is automatically identified and analyzed, in accordance with the disclosure hereinbelow. Alternatively or additionally, one or more additional views (such as a subcostal view, a right parasternal, and/or a suprasternal view) are automatically identified and analyzed.

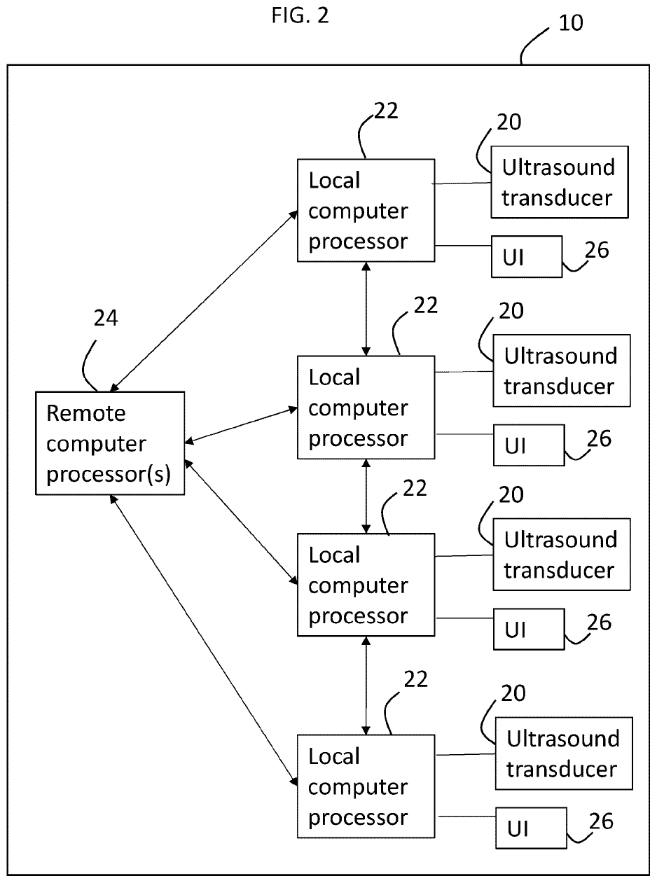

Reference is now made to FIG. 2, which is a block diagram showing portions of an automated echocardiographic image analysis system 10, in accordance with some applications of the present invention. Typically, the system includes a plurality of ultrasound transducers 20 and local computer processors 22, as well as one or more remote computer processors 24, the computer processors being in communication with each other via a network. For some applications, the system includes a plurality of local user interface devices 26. For example, the user interfaces may include an input device, such as a keyboard, a mouse, a joystick, a touchscreen device (such as a smartphone or a tablet computer), a touchpad, a trackball, a voice-command interface, and/or other types of input devices that are known in the art. Alternatively or additionally, the user interface devices include an output device, such as a display, speakers, headphones, a smartphone, or a tablet computer, and/or a printer.

Typically, a user acquires echocardiographic images of a patient using the ultrasound transducer. For some applications, the user manually inputs data regarding the patient and/or extracts data regarding the patient (e.g., from the patient's medical records) for use by the system, using the user interface device. The patient's echocardiographic images are analyzed by one or more of the computer processors, using the analysis techniques described herein. For some applications, based upon the analysis of the patient's echocardiographic images, an output is generated via the user interface device. For example, parameters of the patent that are determined based on the echocardiographic images may be outputted, and/or predictions regarding the patient based on the echocardiographic images may be outputted.

Using prior art techniques it is typically the case that a technician is required to acquire echocardiographic image loops from respective views and then select the optimal echocardiographic image frames from the echocardiographic image loops for performing measurements on the patient's heart. Typically, using the techniques described herein, an entire stream of echocardiographic images, which are acquired from a plurality of different views, is provided as an input to the system. The system is trained to automatically identify image loops that correspond to respective views, to select optimum images and/or image loops for analysis, to extract data from the images and/or image loops, to identify patterns in the data, to extract parameters relating to the patient, and/or to generate predictions regarding patients. Typically, during an initial training stage, the computer processors are trained to perform the above-described steps using video streams of echocardiographic images of many patients. In a subsequent analysis stage, the computer processors are configured to apply patterns that were learned during the training stage to the analysis of a video stream of echocardiographic images of a particular patient in order to perform the above-described steps with respect to the patient. Typically, the echocardiographic images of at least some of (and typically all of) the patients whose echocardiographic images are analyzed by the system in the analysis stage are also utilized for ongoing training of the system. In this manner, training of the system is continuous and is improved over time as the system continues to be provided with additional echocardiographic images and data.

Typically, system 10 analyzes echocardiographic images that do not include Doppler echocardiographic images (i.e., images that indicate blood flow, which are acquired using pulsed-wave or continuous-wave Doppler ultrasound). For some applications, the system analyzes Doppler echocardiographic images in addition to analyzing non-Doppler images.

Typically, during the training stage, the system is trained using a combination of manual inputs (e.g., manual tagging of echocardiographic image loops to identify echocardiographic image loops that correspond to respective views) and artificial-intelligence algorithms. Typically, the artificial-intelligence algorithms train the system to recognize patterns in echocardiographic images such as to identify echocardiographic image loops that correspond to respective views, to select optimum images and/or image loops for analysis, to extract data from the images and/or image loops, to identify patterns in the data, to extract parameters relating to the patient, and/or to generate predictions regarding patients. For some applications, the artificial-intelligence algorithms include a neural network (e.g., a convolutional neural network), a Linear Regression, Logistic Regression, Decision Tree, Support Vector Machine, Naive Bayes, kNN, K-Means, Random Forest, Dimensionality Reduction, and/or Gradient Boosting algorithm. For some applications, the artificial-intelligence algorithms include a visual-transformer algorithm, which typically divides an echocardiogram into fixed-size patches, embeds each of patches, and includes positional embedding as an input to a transformer encoder.

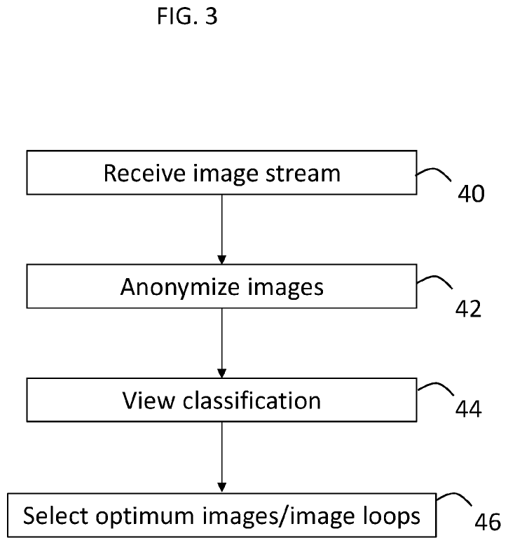

Reference is now made to FIG. 3, which is a flowchart showing steps that are performed during a training stage, in accordance with some applications of the present invention. As noted above, typically, during the training stage, the system is trained using a combination of manual inputs and artificial-intelligence algorithms. Typically, in a first step 40, the computer processors receive a stream of echocardiographic images that are acquired from a plurality of different views. In step 42, the computer processors automatically anonymize the echocardiographic images by identifying any identifying information within the echocardiographic images and removing or obscuring the information. In step 44, view classification is performed, whereby echocardiographic images are tagged as corresponding to respective views. In step 46, optimum echocardiographic images and/or echocardiographic image loops to be used for further analysis are selected. It is noted that some of the above steps are optional, and the scope of the present disclosure includes performing only a portion of these steps.

Typically, step 44 is performed using a combination of artificial-intelligence algorithms (e.g. by the system learning to recognize which echocardiographic images and/or echocardiographic image loops correspond to which views, by identifying features and/or patterns in echocardiographic images and/or echocardiographic image loops) and manual inputs (e.g., by a technician tagging echocardiographic images and/or echocardiographic image loops as corresponding to respective views). As noted above, the system typically includes a plurality of local computer processors 22, as well as one or more remote computer processors 24, the computer processors being in communication with each other via a network. In accordance with respective applications of the present invention, the training and analysis steps that are described herein are performed by the local computer processor, the remote computer processor(s), or a combination thereof. For some applications, data from the local computer processors are communicated to the remote computer processor(s) and the data are fed into an artificial-intelligence algorithm that is run at the remote computer(s). Alternatively or additionally, the local computer processors perform at least some of the analysis of the data using an artificial-intelligence algorithm.

It is noted that some views are typically more difficult to identify than some other views. For some applications, in step 44, for views that are more difficult to identify, a greater number of such echocardiographic images are sent by the system for manual tagging. For example, in an initial step, the system may associate a prediction score with each of the views, the prediction score being indicative of the ability of the system to automatically classify echocardiographic images as belonging to the respective views. For views having a relatively low prediction score, the system sends a greater number of echocardiographic images that are suspected as belonging to those views for manual tagging, whereas for views having a relatively high prediction score, the system sends a lower number of echocardiographic images that are suspected as belonging to those views for manual tagging.

For some applications, in step 44, individual echocardiographic images are tagged as corresponding to respective views. Alternatively or additionally, echocardiographic image loops are tagged as corresponding to respective views. Typically, by tagging echocardiographic image loops rather than individual echocardiographic images during the training stage, a greater number of useful echocardiographic images (e.g., echocardiographic images that can be classified) are provided for learning by the system, but also a greater number of noisy echocardiographic images (e.g., echocardiographic images that are difficult to classify) are provided. For some applications, providing a greater number of noisy echocardiographic images to the system during the training stage trains the system to identify which echocardiographic images are useful for the purpose of the view classification, and/or trains the system to perform view classification even on noisy images.

For some applications, a combination of individual echocardiographic images as well as echocardiographic image loops are used for view classification and/or for selecting optimum echocardiographic images to use for the analysis of a given view. For example, the system may predict that each individual echocardiographic image is likely to correspond to one or more views and to assign a respective confidence score for each of the predictions for each of the echocardiographic images, the confidence score being indicative of the confidence of the system that the prediction for the echocardiographic image is correct. Typically, the system performs the aforementioned analysis with respect to a plurality of echocardiographic images belonging to an echocardiographic image loop. Subsequently, the system typically analyzes the echocardiographic images belonging to the echocardiographic image loop in combination with each other. For example, the system may analyze which view is most likely to be shown in the echocardiographic image loop by analyzing which view has the highest confidence score over all of the echocardiographic images (or over a subset of echocardiographic images) within the image loop. Alternatively or additionally, the system may select which echocardiographic images to use for view classification purposes, based on the confidence score associated with each of the echocardiographic images. For some applications, echocardiographic images having confidence scores that are below a threshold are rejected from being used in further analysis.

For some applications, the system applies augmentation algorithms in order to augment the system's ability to automatically perform view classification. For example, during the training stage, the system may randomly erase portions of at least some of the echocardiographic images. Typically, this trains the system not to rely on particular portions of the echocardiographic images in order to perform view classification, but rather to have the ability to perform view classification with less dependency on which portions of the patient's anatomy or other features are visible within the echocardiographic images. For some applications, the system randomly alters a parameter of at least some of the echocardiographic images and/or portions of at least some of the echocardiographic images. For example, the system may alter the contrast and/or brightness of at least some of the echocardiographic images and/or portions of at least some of the echocardiographic images. Typically, this trains the system not to rely on echocardiographic images being of a given quality in order to perform the view classification, but rather to have the ability to perform view classification with less dependency on the quality and/or particular parameters of the echocardiographic images.

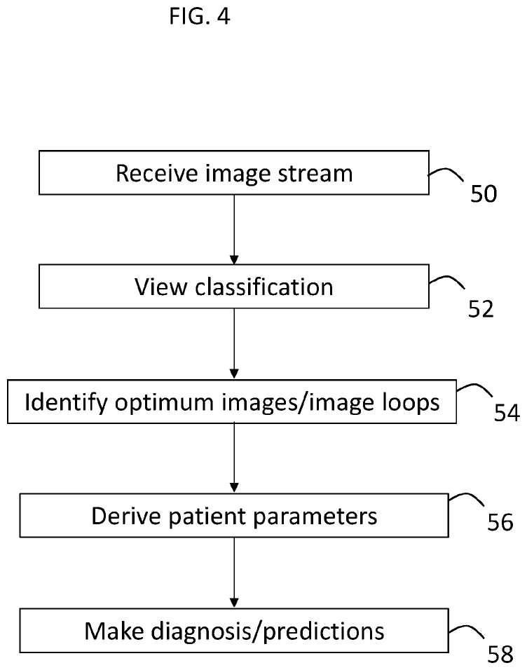

FIG. 4 is a flowchart showing echocardiographic image analysis steps that are performed in accordance with some applications of the present invention. Typically, the steps shown in FIG. 4 are performed during the analysis stage. As described hereinabove, during the analysis stage, the computer processors typically apply patterns that were learned during the training stage to the analysis of a video stream of echocardiographic images of a particular patient in order to identify images and/or image loops that correspond to respective views, to select optimum images and/or image loops for analysis, to extract data from the images and/or image loops, to identify patterns in the data, to extract parameters relating to the patient, and/or to generate predictions regarding the patient.

Typically, in a first step 50, the computer processors receive a stream of echocardiographic images of the patient's heart that are acquired from a plurality of different views. In step 52, view classification is performed, whereby echocardiographic images and/or echocardiographic image loops are classified as corresponding to respective views. In step 54, the system identifies the optimum image echocardiographic images and/or echocardiographic image loops to use for further analysis by the system. In step 56, the identified echocardiographic images and/or echocardiographic image loops are analyzed in order to derive parameters relating to the patient's anatomy and/or physiology. In step 58, the system diagnoses the patient, and/or makes predictions regarding the patient by analyzing the patient's echocardiographic images. It is noted that some of the above steps are optional, and the scope of the present disclosure includes performing only a portion of these steps.

With respect to step 52, typically the view classification is performed based upon the training as described hereinabove with reference to FIG. 3.

As described above, in step 54, the system typically identifies the optimum echocardiographic images and/or echocardiographic image loops to use for further analysis by the system. For some applications, the system is configured to analyze all of the classified echocardiographic images and/or echocardiographic image loops as a whole to determine which combination of echocardiographic images and/or echocardiographic image loops from respective views to use for further analysis, such as to optimize the ability of the system to perform the further steps of analysis. For example, if the patient is undergoing an examination in order to determine the progression of a particular condition, the system may analyze all of the classified echocardiographic images and/or echocardiographic image loops as a whole to determine which combination of echocardiographic images and/or echocardiographic image loops from respective views to use for further analysis, such as to optimize the ability of the system to automatically determine the progression of the patient's condition from the echocardiographic images and/or echocardiographic image loops. Alternatively or additionally, the system randomly selects which combination of echocardiographic images and/or echocardiographic image loops to use for further analysis of respective views. Typically, this increases the reliability of the analysis, by ensuring that the system hasn't been trained to look for a particular type of echocardiographic images and/or echocardiographic image loops for any given view. For some applications, the system randomly removes some image frames from being used for further analysis. Typically, this increases the reliability of the analysis, by ensuring that the system doesn't become reliant on a particular type of echocardiographic image and/or echocardiographic image loop for being used in the further analysis.

For some applications, the system is configured to prevent itself from becoming overly reliant on a particular type of image. For example, if the patient is undergoing an examination in order to determine the progression of a particular condition, a particular type of image of a particular view may be assumed to provide features that are relevant. For some applications, in such cases the system selects echocardiographic images and/or echocardiographic image loops acquired from other views and/or showing other features, in order to allow the system to identify additional features that may be associated with the progression of the condition that may not even be known.

As noted above, in step 56, the echocardiographic images and/or echocardiographic image loops are typically analyzed in order to derive parameters relating to the patient's anatomy and/or physiology. For example, the system may derive the size and shape of the patient's heart, the location and extent of any tissue damage, an assessment of valve function, pumping capacity, the patient's cardiac output, ejection fraction, etc.

For some applications, features are derived from the image loops by analyzing a combination of individual echocardiographic images, and analyzing the sequence of images in the image loop as a time series. For some such applications, features are derived from individual echocardiographic images (for example, by analyzing the image loop at a fixed frequency), but the features from temporally adjacent (or temporally proximate) echocardiographic images are then combined such that changes over the course of the time series are extracted from the image loop. Typically, in this manner, the system extracts dynamic features from the echocardiographic images. For some applications, in the analysis of the image loops as a time series, one or more of the image loops are started from a plurality of different starting points. In this manner, the dataset that is analyzed is artificially increased.

For some applications, in step 56, the system use multi-view analysis, whereby as an alternative to or in addition to extracting features from respective views, the system analyzes combinations of two or more views in combination with each other in order to extract features.

For some applications, two or more image loops from any given view are analyzed and compared with each other, with features typically being extracted from a combination of the image loops, or an optimum image loop from which to extract the features being selected based on the comparison.

For some applications, in step 56, the system use multi-target analysis, whereby as an alternative to or in addition to extracting individual features from an echocardiographic image and/or echocardiographic image loop, the system extracts a plurality of features from an echocardiographic image and/or echocardiographic image loop in parallel with each other. For some applications, extracting a plurality of features in parallel with each other increases the reliability of analysis of the features and/or predictions that are made based upon the features, particularly if the features are related to each other (e.g., if the two features are both indicative of a particular condition, and/or are otherwise correlated with each other). Alternatively or additionally, extracting a plurality of features in parallel with each other increases the processing capabilities and/or speed of the system.

For some applications, in step 56, in addition to deriving parameters relating to the patient's anatomy and/or physiology from the echocardiographic image and/or echocardiographic image loop, the system determines which features or patterns within the images led to the derivation of those parameters. As described hereinabove, for some applications, the artificial-intelligence algorithms include a visual transformer algorithm, which typically divides an echocardiogram into fixed-size patches, embeds each of patches, and includes positional embedding as an input to a transformer encoder. For some applications, the use of a visual transformer algorithm facilitates explainability of the derivations, i.e., the knowledge of what different nodes within the model represent and the importance of respective nodes to the model's performance.

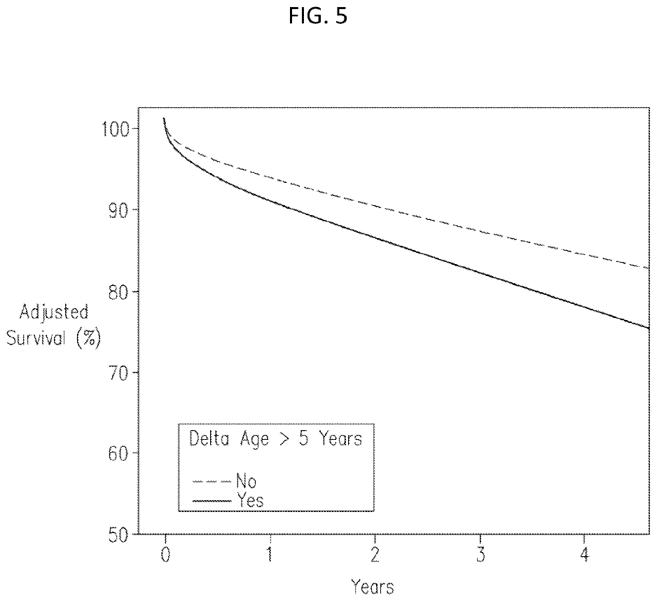

In step 58, the system diagnoses the patient and/or makes predictions regarding the patient by analyzing the patient's echocardiographic images. Typically, the system is configured to determine findings regarding the patient and/or to make predictions regarding the patient by identifying patterns in the echocardiographic images and/or echocardiographic image loops, even in cases in which correlations between features that are evident in the images and such findings and/or such predictions are not known using prior at techniques. For example, the system may analyze the patient's echocardiographic images and to derive the patient's gender or age from the images, even though using prior art techniques there are no particular features within an echocardiogram that are known to enable such as determination, as described in further detail hereinbelow with reference to FIG. 5.

For some applications, the system determines that the patient is suffering from one or more of myocardial infarction, an aortic aneurysm, amyloidosis, dilated cardiomyopathy, hypertrophic cardiomyopathy, pulmonary hypertension, pulmonary emboli, atherosclerosis, aortic stenosis, mitral regurgitation, aortic regurgitation, left ventricular enlargement, and/or left ventricular hypertrophy. For some applications, the system determines that the patient is a candidate for a valve intervention, or an alternative treatment. For some applications, the system determines one or more cardiac or cardiac-related parameters, such as left ventricular ejection fraction, left ventricular function, interventricular septum thickness, left ventricular diastolic function, right ventricular function, and/or right ventricular diastolic function. Alternatively or additionally, the system predicts the likelihood of the patient undergoing a clinical episode. Further alternatively or additionally, the system determines that the patient has a condition that affects a portion of the patient's body other than the patient's heart (such as a renal condition, a hepatic condition, a lymphatic condition, ovarian cancer etc.) , by analyzing the patient's echocardiographic images.

For some applications, in step 58, in addition to making predictions regarding the patient and/or diagnosing the patient, by analyzing the patient's echocardiographic images, the system determines which features or patterns within the images led to the predictions. As described hereinabove, for some applications, the artificial-intelligence algorithms include a visual transformer algorithm, which typically divides an echocardiogram into fixed-size patches, embeds each of patches, and includes positional embedding as an input to a transformer encoder. For some applications, the use of a visual transformer algorithm facilitates explainability of the predictions, i.e., the knowledge of what different nodes represent within the model and the importance of respective nodes to the model's performance.

In some applications, in the event that the system is unable to derive patient parameters (in step 56) and/or is unable to make a diagnosis or predictions (in step 58) to a given level of certainty, the system generates an output indicating that this is the case. For example, the system may generate an output indicating that the results are “indecisive” or recommending that a further scan is performed on the patient. For some applications, the system generates a confidence score with respect to a derived patient parameter, a diagnosis, and/or a prediction. The system typically generates the aforementioned output in response to the confidence score being below a threshold. Typically, this generates greater confidence among medical staff with respect to the system. In this manner, when the system does generate a decisive output, the output may be relied upon by the medical staff.

Reference is now made to FIG. 5, which is a graph with curves indicating, respectively, (a) survival rates of patients whose cardiac-state age differed from their chronological age by more than 5 years, and (b) survival rates of patients whose cardiac-state age did not differ from their chronological age by more than 5 years, as determined in accordance with some applications of the present invention. The data shown in FIG. 5 is based upon a study that was conducted in order to determine whether machine learning algorithms could be trained to predict a patient's cardiac-state age and cardiac-state sex using transthoracic standard echocardiography, whereby cardiac-state age and cardiac-state sex are indicative of the age that the patient's heart appears to be, based upon the analysis of the of stream of echocardiographic images, and which sex the patient appears to be based upon the analysis of the of stream of echocardiographic images. It was hypothesized that cardiac-state age and cardiac-state sex would be superior to chronological age and biological sex in predicting long-term survival.

The analysis was based on 122,014 unique patients who underwent transthoracic standard echocardiography evaluation between 2007 and 2021 at Sheba Medical Center, Ramat Gan, Israel. Survival data was available for all subjects from the Israeli Population Register. Three mutually exclusive cohorts were used for training (N=76,342[62%]), validation (N=22,825[19%]) and testing (N=22,847[19%]). Patients were dichotomized into two mutually exclusive groups. The first group included patients whose cardiac-state age was greater than their chronological age by more than 5 years. The second group included all other patients. A Multivariate Cox regression model was used to investigate the association between cardiac-state age and chronological age with overall survival.

The final test cohort included 18,447 unique patients of whom 10,640(58%) were men and with a median age of 66 (IQR 76-54). Age was estimated as a continuous variable with an average error of 4.89 years. The analysis demonstrated a Root Mean Square Error (RMSE) of 6.327 and Pearson correlation coefficient of 0.922. Sex was estimated as a probabilistic value with total accuracy of 96.1% and an area under the curve of 0.993. During a median follow-up of 4.3 (IQR, 2.1-6.3) years, 5147 (27.9%) patients died. As indicated by the graph in FIG. 5, the Multivariate Cox regression model with adjustment for age, sex and ejection fraction demonstrated that having a cardiac-state age that was greater than patients' chronological age by more than 5 years was associated with an independent and significant 50% increased risk of death during follow up (95% CI 1.4-1.6, p<0.001). Similarly, biological females who were determined to be cardiac-state females was associated with a significant 30% increased risk of death in the same model (95% CI 1.2-1.4, p<0.001).

The above-described results indicate that applying artificial intelligence to the analysis of echocardiographic images allows prediction of patient's cardiac-state age and sex, with these parameters being effective predictors of overall survival rates. In accordance with the above-described results, cardiac-state age and sex can also be used for risk stratification, patient outcome predications, estimation of biological age, etc.

In accordance with the above-described results, for some applications, based upon the analysis of echocardiographic images, the system determines a cardiac-state age of the patient, the cardiac-state age of the patient being indicative of the age that the patient's heart appears to be, based upon the analysis of the of stream of echocardiographic images (and, thus, indicative of a risk of the patient suffering from cardiac conditions). For some applications, the system receives an input indicating the patient's chronological age, and makes a prediction regarding the patient based upon a difference between the patient's chronological age and the patient's cardiac-state age. For some applications, based upon the analysis of echocardiographic images, the system determines a cardiac-state sex of the patient, the cardiac-state sex of the patient being indicative of the sex that the patient appears to be, based upon the analysis of the of stream of echocardiographic images (and, thus, indicative of a risk of the patient suffering from cardiac conditions). For some applications, the system receives an input indicating the patient's biological sex, and makes a prediction regarding the patient based upon a difference between the patient's biological sex and the patient's cardiac-state sex.

Although some applications of the present disclosure have been described as being applied to echocardiographic images, the scope of the disclosure includes applying generally similar techniques to echo images of a different portion of the subject's anatomy (e.g., the subject's lungs, reproductive system, gastrointestinal tract, etc.) and/or to images acquired in a different imaging modality (such as MRI, X-ray, CT, PET, etc.), mutatis mutandis.

Applications of the invention described herein can take the form of a computer program product accessible from a computer-usable or computer-readable medium (e.g., a non-transitory computer-readable medium) providing program code for use by or in connection with a computer or any instruction execution system, such as computer processors 22, 24. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processors 22, 24) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processors 22, 24) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processors 22,24 are typically hardware devices programmed with computer program instructions to produce a special purpose computer. For example, when programmed to perform the algorithms described with reference to the Figures, computer processors 22,24 typically acts as a special purpose image-analysis computer processor. Typically, the operations described herein that are performed by computer processors 22, 24 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
at least one computer processor configured to:
receive a stream of echocardiographic images of a patient's heart that are acquired from a plurality of different views;
analyze the stream of echocardiographic images; and
determine a cardiac-state age of the patient based upon the analysis, the cardiac-state age of the patient being indicative of an age that the patient's heart appears to be, based upon the analysis of the stream of echocardiographic images.

2. The apparatus according to claim 1, wherein the at least one computer processor is configured to receive an input indicating the patient's chronological age, and wherein the computer processor is configured to make a prediction regarding the patient based upon a difference between the patient's chronological age and the patient's cardiac-state age.

3. The apparatus according to claim 1, wherein in response to determining that it is not possible to determine the cardiac-state age of the patient based upon the analysis a given degree of certainty, the computer processor is configured to generate an output indicating that this is the case.

4. The apparatus according to claim 1, wherein the at least one computer processor is configured to diagnose the patient and/or make a prediction regarding the patient at least partially based upon the analysis of the stream of echocardiographic images.

5. The apparatus according to claim 1, wherein the at least one computer processor is configured to:
analyze the stream of echocardiographic images using a visual-transformer algorithm;
diagnose the patient and/or make a prediction regarding the patient based upon the analysis; and
generate an output indicating the diagnosis and/or prediction, and indicating which features and/or patterns within the stream of echocardiographic images of the patient's heart that led to the diagnosis and/or prediction.

6. The apparatus according to any one of claims 1-5, wherein the at least one computer processor is configured to:
classify the echocardiographic images and/or loops of the echocardiographic images as corresponding to respective views;
perform further analysis of a portion of the echocardiographic images and/or echocardiographic image loops that correspond to one more views; and
determine the cardiac-state age of the patient based upon the based upon the further analysis of the portion of the echocardiographic images and/or echocardiographic image loops that correspond to one more views.

7. The apparatus according to claim 6, wherein at least some of the echocardiographic images and/or echocardiographic image loops that correspond to one more views that are selected for the further analysis are randomly selected.

8. The apparatus according to claim 6, wherein at least some of the echocardiographic images and/or echocardiographic image loops are randomly removed from being used for the further analysis of the portion of the echocardiographic images and/or echocardiographic image loops that correspond to one more views.

9. The apparatus according to claim 6, wherein the at least one computer processor is configured to:
select one or more echocardiographic images and/or echocardiographic image loops within the stream of echocardiographic images;
for each of the one or more selected echocardiographic images and/or echocardiographic image loops, perform multi-target analysis by extracting a plurality of features from the one or more echocardiographic images and/or echocardiographic image loops in parallel with each other; and
diagnose the patient and/or make a prediction regarding the patient at least partially based upon the plurality of features that are extracted from the one or more echocardiographic images and/or echocardiographic image loops.

10. The apparatus according to any one of claims 1-5, wherein the at least one computer processor is configured to:
derive features from individual echocardiographic images;
combine features from temporally proximate echocardiographic images within a loop of echocardiographic images such that changes over time are extracted from the loop of echocardiographic images; and
diagnose the patient and/or make a prediction regarding the patient at least partially based upon the changes over time that are extracted from the loop of echocardiographic images.

11. The apparatus according to claim 10, wherein the at least one computer processor is configured to define a plurality of loops of echocardiographic images with each of the loops starting from a different starting point, and wherein the at least one computer processor is configured to combine features from temporally proximate echocardiographic images within each of the plurality of loops of echocardiographic images.

12. The apparatus according to any one of claims 1-5, wherein the at least one computer processor is configured to determine a cardiac-state sex of the patient, the cardiac-state sex of the patient being indicative of a sex that the patient appears to be based upon the analysis of the of stream of echocardiographic images.

13. The apparatus according to claim 12, wherein the computer processor is configured to receive an input indicating the patient's biological sex, and wherein the computer processor is configured to make a prediction regarding the patient based upon a difference between the patient's biological sex and the patient's cardiac-state sex.

14. A method comprising:
using at least one computer processor:
receiving a stream of echocardiographic images of a patient's heart that are acquired from a plurality of different views;
analyzing the stream of echocardiographic images; and
determining a cardiac-state age of the patient based upon the analysis, the cardiac-state age of the patient being indicative of an age that the patient's heart appears to be, based upon the analysis of the stream of echocardiographic images.

15. A computer software product, for use with a blood sample, the computer software product comprising a non-transitory computer-readable medium in which program instructions are stored, which instructions, when read by a computer cause the computer to perform the steps of:
receiving a stream of echocardiographic images of a patient's heart that are acquired from a plurality of different views;
analyzing the stream of echocardiographic images; and
determining a cardiac-state age of the patient based upon the analysis, the cardiac-state age of the patient being indicative of an age that the patient's heart appears to be, based upon the analysis of the stream of echocardiographic images.

16. Apparatus comprising:
at least one computer processor configured to:
receive a stream of echocardiographic images of a patient's heart that are acquired from a plurality of different views;
classify echocardiographic images and/or loops of the echocardiographic images as corresponding to respective views;
perform further analysis of a portion of the echocardiographic images and/or echocardiographic image loops that correspond to one more views, wherein at least some of the echocardiographic images and/or echocardiographic image loops that correspond to one more views that are selected for the further analysis are randomly selected; and
diagnose the patient and/or make a prediction regarding the patient at least partially based upon the further analysis of the portion of the echocardiographic images and/or echocardiographic image loops that correspond to one more views.

17. The apparatus according to claim 16, wherein in response to determining that it is not possible to diagnose the patient and/or make a prediction regarding the patient to a given degree certainty, the computer processor is configured to generate an output indicating that this is the case.

18. The apparatus according to claim 16, wherein at least some of the echocardiographic images and/or echocardiographic image loops are randomly removed from being used for the further analysis of the portion of the echocardiographic images and/or echocardiographic image loops that correspond to one more views.

19. The apparatus according to claim 16, wherein the at least one computer processor is configured to:
select one or more echocardiographic images and/or echocardiographic image loops from within the stream of echocardiographic images;
for each of the one or more selected echocardiographic images and/or echocardiographic image loops, perform multi-target analysis by extracting a plurality of features from the one or more echocardiographic images and/or echocardiographic image loops in parallel with each other; and
diagnose the patient and/or make a prediction regarding the patient at least partially based upon the plurality of features that are extracted from the one or more echocardiographic images and/or echocardiographic image loops.

20. The apparatus according to claim 16, wherein the at least one computer processor is configured to:
analyze the stream of echocardiographic images using a visual-transformer algorithm;
diagnose the patient and/or make a prediction regarding the patient based upon the analysis; and
generate an output indicating the diagnosis and/or prediction, and indicating which features and/or patterns within the stream of echocardiographic images of the patient's heart that led to the diagnosis and/or prediction.

21. The apparatus according to any one of claims 16-20, wherein the at least one computer processor is configured to:
derive features from individual echocardiographic images;
combine features from temporally proximate echocardiographic images within a loop of echocardiographic images such that changes over time are extracted from the loop of echocardiographic images; and
diagnose the patient and/or make a prediction regarding the patient at least partially based upon the changes over time that are extracted from the loop of echocardiographic images.

22. The apparatus according to claim 21, wherein the at least one computer processor is configured to define a plurality of loops of echocardiographic images, with each of the loops starting from a different starting point, and wherein the at least one computer processor is configured to combine features from temporally proximate echocardiographic images within each of the plurality of loops of echocardiographic images.

23. The apparatus according to any one of claims 16-20, wherein the at least one computer processor is configured to determine a cardiac-state age of the patient, the cardiac-state age of the patient being indicative of an age that the patient's heart appears to be, based upon the further analysis of the portion of the echocardiographic images and/or echocardiographic image loops that correspond to one more views.

24. The apparatus according to claim 23, wherein the computer processor is configured to receive an input indicating the patient's chronological age, and wherein the computer processor is configured to make a prediction regarding the patient based upon a difference between the patient's chronological age and the patient's cardiac-state age.

25. The apparatus according to any one of claims 16-20, wherein the at least one computer processor is configured to determine a cardiac-state sex of the patient, the cardiac-state sex of the patient being indicative of a sex that the patient appears to be, based upon the further analysis of the portion of the echocardiographic images and/or echocardiographic image loops that correspond to one more views.

26. The apparatus according to claim 25, wherein the computer processor is configured to receive an input indicating the patient's biological sex, and wherein the computer processor is configured to make a prediction regarding the patient based upon a difference between the patient's biological sex and the patient's cardiac-state sex.

27. A method comprising:
using at least one computer processor:
receiving a stream of echocardiographic images of a patient's heart that are acquired from a plurality of different views;
classifying echocardiographic images and/or loops of the echocardiographic images as corresponding to respective views;
performing further analysis of a portion of the echocardiographic images and/or echocardiographic image loops that correspond to one more views, wherein at least some of the echocardiographic images and/or echocardiographic image loops that correspond to one more views that are selected for the further analysis are randomly selected; and
diagnosing the patient and/or making a prediction regarding the patient at least partially based upon the further analysis of the portion of the echocardiographic images and/or echocardiographic image loops that correspond to one more views.

28. A computer software product, for use with a blood sample, the computer software product comprising a non-transitory computer-readable medium in which program instructions are stored, which instructions, when read by a computer cause the computer to perform the steps of:
receiving a stream of echocardiographic images of a patient's heart that are acquired from a plurality of different views;
classifying echocardiographic images and/or loops of the echocardiographic images as corresponding to respective views;
performing further analysis of a portion of the echocardiographic images and/or echocardiographic image loops that correspond to one more views, wherein at least some of the echocardiographic images and/or echocardiographic image loops that correspond to one more views that are selected for the further analysis are randomly selected; and
diagnosing the patient and/or making a prediction regarding the patient at least partially based upon the further analysis of the portion of the echocardiographic images and/or echocardiographic image loops that correspond to one more views.

29. Apparatus comprising:
at least one computer processor configured to:
receive a stream of echocardiographic images of a patient's heart that are acquired from a plurality of different views;
derive features from individual echocardiographic images;
combine features from temporally proximate echocardiographic images within a loop of echocardiographic images such that changes over time are extracted from the loop of echocardiographic images; and
diagnose the patient and/or make a prediction regarding the patient at least partially based upon the changes over time that are extracted from the loop of echocardiographic images.

30. The apparatus according to claim 29, wherein the at least one computer processor is configured to define a plurality of loops of echocardiographic images, with each of the loops starting from a different starting point, and wherein the at least one computer processor is configured to combine features from temporally proximate echocardiographic images within each of the plurality of loops of echocardiographic images.

31. The apparatus according to claim 29, wherein in response to determining that it is not possible to diagnose the patient and/or make a prediction regarding the patient to a given degree certainty, the computer processor is configured to generate an output indicating that this is the case.

32. The apparatus according to claim 29, wherein the at least one computer processor is configured to:
analyze the stream of echocardiographic images using a visual-transformer algorithm;
diagnose the patient and/or make a prediction regarding the patient based upon the analysis; and
generate an output indicating the diagnosis and/or prediction, and indicating which features and/or patterns within the stream of echocardiographic images of the patient's heart that led to the diagnosis and/or prediction.

33. The apparatus according to any one of claims 29-32, wherein the at least one computer processor is configured to:
classify the echocardiographic images and/or loops of the echocardiographic images as corresponding to respective views;
analyze a portion of the echocardiographic images and/or echocardiographic image loops that correspond to one more views; and
diagnose the patient and/or make a prediction regarding the patient at least partially based upon analyzing the portion of the echocardiographic images and/or echocardiographic image loops that correspond to one more views.

34. The apparatus according to claim 33, wherein at least some of the echocardiographic images and/or echocardiographic image loops that correspond to one more views that are selected for analyzing are randomly selected.

35. The apparatus according to claim 33, wherein at least some of the echocardiographic images and/or echocardiographic image loops are randomly removed from being used for analyzing the portion of the echocardiographic images and/or echocardiographic image loops that correspond to one more views.

36. The apparatus according to claim 33, wherein the at least one computer processor is configured to:
select one or more echocardiographic images and/or echocardiographic image loops within the stream of echocardiographic images;
for each of the one or more selected echocardiographic images and/or echocardiographic image loops, perform multi-target analysis by extracting a plurality of features from the one or more echocardiographic images and/or echocardiographic image loops in parallel with each other; and
diagnose the patient and/or make a prediction regarding the patient at least partially based upon the plurality of features that are extracted from the one or more echocardiographic images and/or echocardiographic image loops.

37. The apparatus according to any one of claims 29-32, wherein the at least one computer processor is configured to determine a cardiac-state age of the patient, the cardiac-state age of the patient being indicative of an age that the patient's heart appears to be based upon the changes over time that are extracted from the loop of echocardiographic images.

38. The apparatus according to claim 37, wherein the computer processor is configured to receive an input indicating the patient's chronological age, and wherein the computer processor is configured to make a prediction regarding the patient based upon a difference between the patient's chronological age and the patient's cardiac-state age.

39. The apparatus according to any one of claims 29-32, wherein the at least one computer processor is configured to determine a cardiac-state sex of the patient, the cardiac-state sex of the patient being indicative of a sex that the patient appears to be based upon the changes over time that are extracted from the loop of echocardiographic images.

40. The apparatus according to claim 39, wherein the computer processor is configured to receive an input indicating the patient's biological sex, and wherein the computer processor is configured to make a prediction regarding the patient based upon a difference between the patient's biological sex and the patient's cardiac-state sex.

41. A method comprising:
using at least one computer processor:
receiving a stream of echocardiographic images of a patient's heart that are acquired from a plurality of different views;
deriving features from individual echocardiographic images;
combining features from temporally proximate echocardiographic images within a loop of echocardiographic images such that changes over time are extracted from the loop of echocardiographic images; and
diagnosing the patient and/or making a prediction regarding the patient at least partially based upon the changes over time that are extracted from the loop of echocardiographic images.

42. A computer software product, for use with a blood sample, the computer software product comprising a non-transitory computer-readable medium in which program instructions are stored, which instructions, when read by a computer cause the computer to perform the steps of:
receiving a stream of echocardiographic images of a patient's heart that are acquired from a plurality of different views;
deriving features from individual echocardiographic images;
combining features from temporally proximate echocardiographic images within a loop of echocardiographic images such that changes over time are extracted from the loop of echocardiographic images; and
diagnosing the patient and/or making a prediction regarding the patient at least partially based upon the changes over time that are extracted from the loop of echocardiographic images.
